(19) United States
(12) Patent Application Publication
(10) Pub. No.: US 2005/0267482 A1
(43) Pub. Date: Dec. 1, 2005

(54) Bone treatment method with implants and instrumentation

(76) Inventor: Edward Robert Hyde JR., Turlock, CA (US)

Correspondence Address: EDWARD R. HYDE JR, 2928 ARNOLDSON AVE, SAN DIEGO, CA, 92122, US

(21) Appl. No.: 11/112,188

(22) Filed: Apr. 22, 2005

Related U.S. Application Data
(60) Provisional application No. 60/521,421, filed on Apr. 22, 2004.

(51) Int. Cl. A61B 17/32

(52) U.S. Cl. 606/79

(57) ABSTRACT

A method, instrumentation and implants for a minimally invasive bone and joint treatments allow cuts in bones to be made simultaneously. This method produces a simple precise alignment of cuts on opposite sides of a joint or bone part. It eliminates many steps need to align the numerous cuts used in current joint replacement and bone treatments. In some cases only one cut will be needed. The cuts can also easily be adapted to different anatomical variation and allows implants to be implanted in a fashion where the implants oriented individually in several different planes. The method and instruments allows for other joint and bone treatments besides joint replacement.

This application claims benefit of provisional application BONE TREATMENT METHOD No. 60/521421 that was filed on Apr. 22, 2004 16:42:16 EDT.

### BACKGROUND OF THE INVENTION

Joint replacements are now very common procedures. There are artificial joints that are partial replacements and there are total joint replacements.

Most joint replacements, especially total joint replacements, require more than one component and the method of implantation requires treatment of a bone or bone part for the implantation of each component. There are usually systems to guide the surgeon in making the necessary cuts and/or other preparations for the placement of each component. Components are typically on each side of the joint space that need to be aligned with the anatomy and aligned relative to each other.

Currently there has been an increased focus on inter-component alignment, giving increased interest to the concept of computer navigation (CN). CN however typically focuses on each component with relation to the bone anatomy rather that specifically the relative positions of one component with the other component or components.

The greater the number of steps needed to complete a portion of a procedure the greater the chances are for error, especially if one step builds on accomplishment of the previous step. Systems that build upon previous steps without references, cross references and checks to the landmarks and measurements of the preceding steps typically compound errors and dilute the utility of the guide system, especially when they do not take the relative component alignment into account. Reducing steps and linking references to anatomical landmarks and providing cross references will improve surgeon performance.

The Bone Treatment method, instrumentation and implants simplify multi-step procedures and reduce errors in implantation for joint replacement and other technically demanding work.

This is accomplished by making bone cuts, especially the first bone cuts substantially part of one step. The cuts are made simultaneously or simultaneously with respect to a group of sequenced cuts where some of the sequenced cuts cut more than one bone.

### SUMMARY OF INVENTION

The surgical procedure for a UKA will be presented briefly. The Bone Treatment Method Technique and Treatment Options will then be discussed in detail. The figures included in the application will substantially concentrate on the method, instruments and implants for a Unicondylar Knee. The method and instruments can be utilized for any joint or bone treatment. The specifics of the implants are for a Unicondylar knee. Many of the features of the UKA implants especially fixation elements can be utilized in other joint and bone applications.

Brief Outline of Standard Unicondylar Knee

1. Positioning of Tibial Cutting Guide and fixation pins. Aligned to surface anatomy of ankle.

2. Tibial cut Two steps

3. Placement of Tibial spacer

4. Placement of Distal Femoral resection guide and fixation pins

5. Distal Femoral Cut

6. Placement of Femoral Post hole

7. Placement of Chamfer Guide

8. Anterior Chamfer Cut

9. Posterior Chamfer Cut

10. Placement of Femoral Fixation Template

11. Placement of Femoral Trial

12. Placement of Tibial Fixation Template

13. Cutting of slot for Tibial keel

14. Cement preparation

15. Implantation of components.

The Bone Treatment Method Technique for Single Compartment Knee Pathology

Initial Cut

1. Position patient's knee in adjustable cradle in slight flexion (approximately 7 degrees) to match tibial AP tilt. (The first cut can also be made with the femur and tibia at approximately 90 degrees or any angle preferred by the surgeon)

2. Small Incision is made centered at the joint line to place the cutting guide device shaft

3. Cutting guide device shaft with soft tissue protector is then inserted and soft tissue protector is deployed (The first cut can terminate before the cut is made all the way through, which would make the soft tissue protector optional)

4. Cutting guide device shaft is oriented to the weight bearing axis through the knee, the mechanical axis of the femur and the tibia, the varus/valgus tilt of the tibia relative to the femur, the AP tilt of the tibia and the rotation in the vertical direction of the femur with respect to the tibia. This can be done anatomically, with guides that determine the mechanical axis or by CN.

5. A computer guided cutting device can be used to orientate the cut to the femur and/or tibia bone anatomy or can be attached directly to the femur and/or tibia to guide the cuts.

6. Patient's anatomy is then matched to their normal anatomy (good leg) or to a preferred mechanical axis (matched to their height, weight and sex) in terms of varus/valgus orientation, AP tilt and rotation

7. Realignment of patient's knee is considered to correct for deformity or anatomy that would produce abnormal kinetics or kinematics. Pre-operative calculations are used to set the guide jig for the cutting device.

8. Alignment of the first projected cut is checked with x-rays, fluoroscopy, computer navigation (CN) and/or ultrasound in more than one plane

9. The guide mechanism is placed over guide pin and secured. Six degrees of freedom correction for alignment of cut is made.

10. The cutting device, which can be a core cutter (annular type cutter) with a cannulated centering drill in the preferred embodiment, is introduced into the cutting guide over the guide shaft and through the obturator in the cutting guide.

The core can be cut by any method that retains the bone cuttings as substantially whole pieces or a drill, bit or bore cutting device can be used.

11. The orientation of the cut is checked in more than one plane.

12. The core cut is made through the distal femur and the proximal tibia in one step.

The core cut is made at low RPM. The wall thickness of the core cutter can be in the range of 0.01 in. to 0.005 in. or smaller if necessary.

The core cut can be continued until the bone is cut all the way through and the protective shield is engaged or it can be terminated before the bone is completely cut through.

13. The core is removed. It will be a composite of: 1) the distal femoral cartilage, cortical bone and cancellous bone and 2) the proximal tibial cartilage, cortical bone and cancellous bone. There will also be other joint and meniscus debris.

14. The core is kept viable and saved for later use.

Treatment Options—Knee.

1. Allograft or Autograft

a. A preferred method is to treat pathology of articular surface, i.e. meniscus, cartilage or bone by know and accepted means is accomplished on the core material and then the treated core composite is reinsert. The bone cut/fracture is secured to the femur/tibia and will proceed on to healing and revascularization of the removed bone.

b. Another preferred method would be to use a fresh frozen composite allograft from a similar core cut that is a composite of femoral cancellous bone, cartilage, intact meniscus with associated tibial cartilage cortical bone and cancellous bone. The Allograft/Autograft is mechanically secured to the patient's native bone.

2. Artificial Joint Resurfacing (AJR)

a. Surface replacements are designed to conserve on bone removal and have modified limited fixation such as a peg instead of a stem. (Copeland Shoulder Resurfacing and the Birmingham, Cormet and Conserve Hip Resurfacings)

b. The upper and/or lower parts of the Bone Treatment Method allograft core can be treated with a resurfacing and re-incorporated as a composite (resurfacing element plus bone or bone and cartilage into the patient. The AJR are typically placed in a non-cemented fashion. Frequently only one side of the joint is treated

c. AJR can also be utilized with bone graft (allograft or autograft) or bone substitutes, bone matrix, BMP, as well as any metal, ceramic or carbon-based matrix of any type of such as a scaffold, lattice or matrix.

3. Magnetic Interposition Arthroplasty (MIA)

a. Magnetic Arrays can be placed in the upper and lower parts of the allograft core and re-incorporated as a composite (magnetic arrays plus bone or bone and cartilage into the patient)

b. MIA can also be utilized with bone graft (allograft or autograft)or bone substitutes, bone matrix, BMP, as well as any metal, ceramic or carbon-based matrix of any type of such as a scaffold, lattice or matrix.

4. Artificial Joint Unicondylar Knee Arthroplasty (UKA)

a. A more conventional prosthesis using more substantial implants than the AJR can be used. Typically there is a femoral component and a tibial component. Either or both can be modular. Fixation is typically more substantial. Typically the components are cemented with PMMA.

b. The upper and/or lower parts of the Bone Treatment Method allograft core can be treated with a resurfacing and re-incorporated as a composite(resurfacing element plus bone or bone and cartilage into the patient

c. UKA can also be utilized with bone graft (allograft or autograft) or bone substitutes, bone matrix, BMP, as well as any metal, ceramic or carbon-based matrix of any type of such as a scaffold, lattice or matrix.

5. Artificial Joint Medial and Lateral Unicondylar Knee Arthroplasties

a. Simultaneous UKAs can be implanted as in 4 above (UKA)

b. The implants can be placed using two sequential independent procedures

6. Total Knee Replacement

a. A modular TKA can be implanted and assembled in vivo or in situ.

b. Two femoral condyle replacements with or without a bridging unit on the femoral side and two tibial articulating surfaces with or without a bridging unit between the tibial articulating surfaces with or without a patella treatment or resurfacing.

Treatment Options other Joints

A. Any other joint or bone part can treated in a like fashion if the clinical situation is appropriate.

B. Other joints include but are not limited to:

1. Temporal Mandibular Joint (TMJ)

2. Acromioclavicular Joint (AC joint)

3. Shoulder

4. Elbow

5. Wrist

6. Carpal/Carpal

7. Carpal/Metacarpal

8. MCP

9. PIP

10. DIP

11. Spine Facet

12. Spine Disc (Amphiarthrosis)

13. SI Joint

14. Hip

15. Knee

16. Ankle

17. Tarsal/Tarsal

18. Tarsal/Metatarsal

19. MTP

C. Bone parts treated in this fashion can include

1. Fractures i. Fresh fractures ii. Non-Union iii. Mal-Union iv. Pseudarthrosis

D. Other bone pathology: 1. Tumors 2. Congenital/Genetic Pathologies 3. Metabolic Bone Disease

### BRIEF DESCRIPTION OF DRAWINGS

FIG. 1 AP/PA Knee with lateral cut position

FIG. 2 Lateral Knee—with lateral cut position

FIG. 3—Axial Views with and without menisci Total Uni-Compartmental Resection

FIG. 4—Axial Views with and without menisci Limited Uni-Compartmental Resection

FIG. 5 AP—with lateral cut position: 3 size cuts

FIG. 6A—AP—with lateral cut position: a size cut

FIG. 6B—AP—with lateral cut position: b size cut

FIG. 6C—AP—with lateral cut position: c size cut

FIG. 7A—AP—with lateral cut position: square shaped cut position

FIG. 7B—AP—with lateral cut position: square shaped cut

FIG. 8—Example of one 6 Degrees Of Freedom medial & lateral cuts (negative of cut without bone)

FIG. 9—AP—with lateral cut position: normal lateral joint line anatomy

FIG. 10—AP—with lateral cut position: normal lateral joint line anatomy restored with implant(Center Line—Asymmetric)

FIG. 11—AP—with medial cut position: varus medial joint line anatomy

FIG. 12A—AP—with medial cut position: varus medial joint line anatomy Restoration of transverse alignment. FIG. 12B—AP—with medial cut position: varus medial joint line anatomy Secondary femoral resection

FIG. 12C-AP—with medial cut position: normal varus joint line anatomy Restoration of joint line height

FIG. 13—AP—with medial cut position: normal varus medial joint line anatomy Implant Correction Instruments

FIG. 14—Dissector/Tissue Protector Type I

FIG. 15—Dissector/Tissue Protector Type I Detail

FIG. 16—Dissector/Tissue Protector Type II

FIG. 17—Initial Cutting Guide—Type I

FIG. 17A—Initial Annular Core Cutting Device

FIG. 18—Joint Line Distracter Type I—Plates

FIG. 19—Joint Line Distracter Type II Balloon, Jack, Wedge, etc.

Implants

FIG. 20A—Cylinder Symmetrical

FIG. 20B—Cylinder Asymmetrical

FIG. 21—Cylinder Symmetrical Curved

FIG. 22—Cylinder Symmetrical Femoral Component

FIG. 23—Cylinder Symmetrical Femoral Component with Flat Tibial Component

FIG. 24—Cylinder Symmetrical Curved Tibial Component

FIG. 25—Cylinder Symmetrical Comparison of two Curved Tibial Components

FIG. 26—Cylinder Symmetrical Curved Tibial Component with peg and fins

FIG. 27—Cylinder Symmetrical Curved Tibial Component with Rebar

FIG. 28—Flat Tibial Component with curved keel

FIG. 29—Flat Tibial Component with curved keel and Magnetic Array

FIG. 30 with Curved Tibial Component

FIG. 31—Cylinder Symmetrical Femoral Component with Flat Tibial Component and curved keel

FIG. 32—Cylinder Mobile Bearing with Fenestrated Fixation

FIG. 33—Cylinder Symmetrical Femoral Component with Fenestrated Fixation

### DETAILED DESCRIPTION

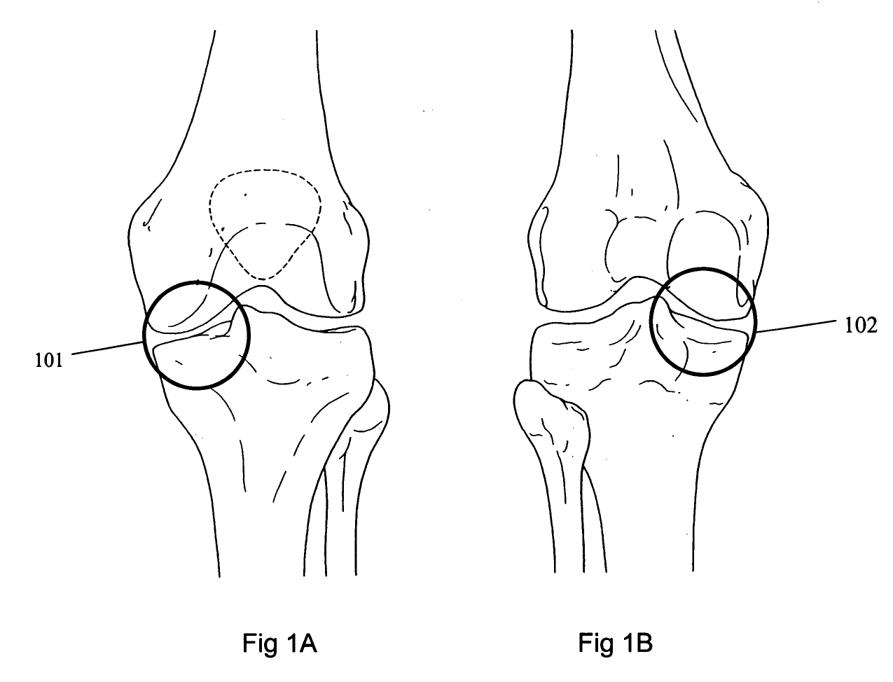

FIG. 1A shows the Anterior-Posterior (AP) position of a medial joint line cut (101) that resects in general the bone of the femur and tibia within the outline. FIG. 1B shows the Posterior-Anterior (PA) position of a medial joint line cut (102) that resects in general the bone of the femur and tibia within the outline.

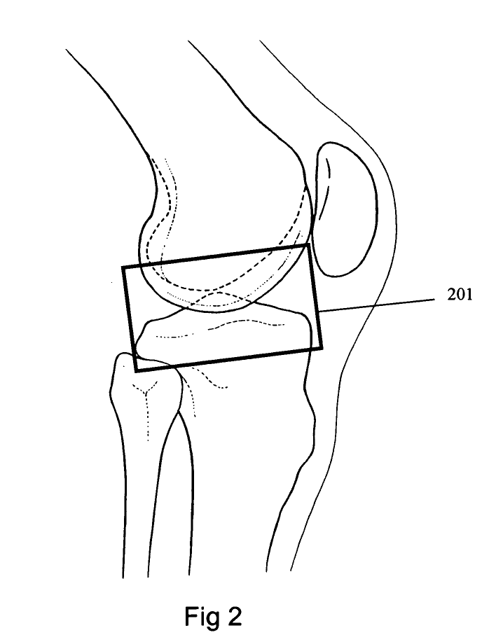

FIG. 2 shows the path of cut (201) from the lateral side (FIG. 1) of the knee showing the general direction of the medial cut.

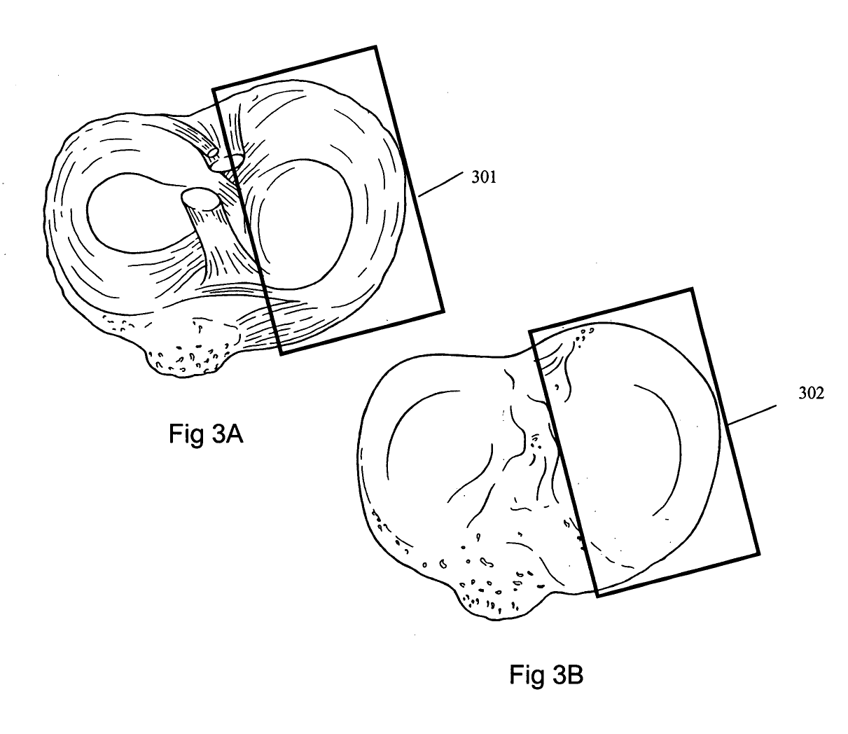

FIG. 3A shows an axial view of the top of the tibia with the menisci in place and the outline of a cut (301) that substantially resects the complete bone and cartilage of the proximal medial tibia. FIG. 3B shows an axial view of the top of the tibia without the menisci in place and the outline of a cut (302) that substantially resects the complete bone and cartilage of the proximal medial tibia.

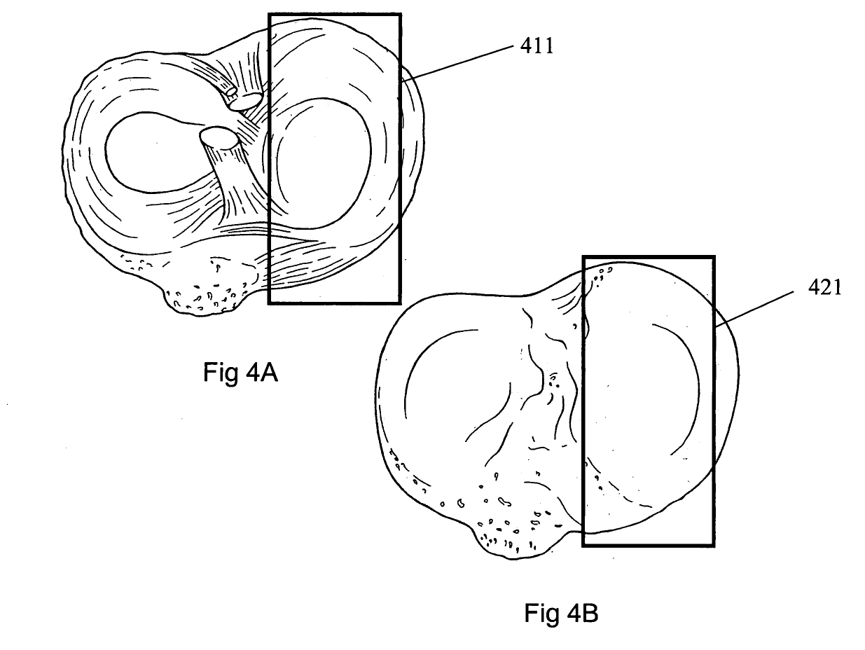

FIG. 4A shows an axial view of the top of the tibia with the menisci in place and the outline of a cut (411) that substantially resects a portion of the bone and cartilage of the proximal medial tibia. FIG. 4B shows an axial of the top of the tibia without the menisci in place and the outline of a cut (421) that substantially resects a portion of the bone and cartilage of the proximal medial tibia.

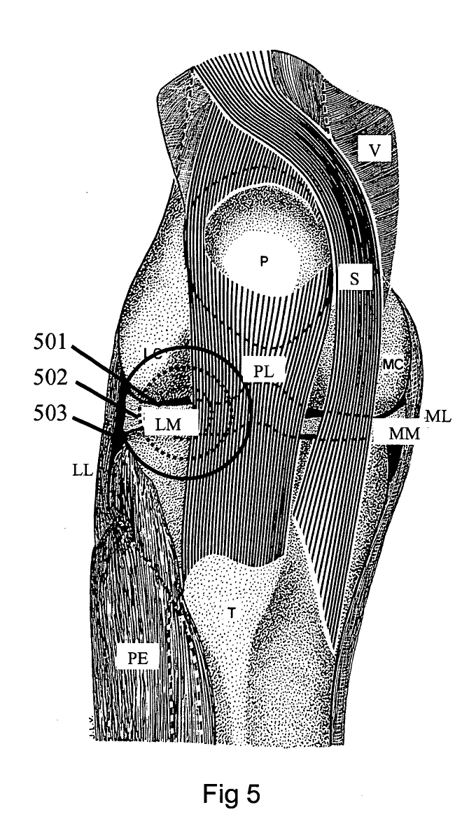

FIGS. 5 through 13 demonstrate variations in cuts at the joint line for the treatment of a knee. The pertinent anatomy has been labeled using letters. (V=Vastus Medialis, P=Patella, S=Sartorious, LC=Lateral Condyle of the femur, MC=Medial Condyle of the femur, PL=Patellar Ligament, LL=Lateral collateral ligament, LM=Lateral Meniscus, MM=Medial Meniscus, ML=Medial collateral ligament, T=Tibia and PE=Peroneal muscles.) These labels are to orient those less familiar with knee anatomy.

The cuts represented by lines indicate the approximate position of the cuts at the joint. The overlay of the lines on soft tissues does not indicate that the cuts go through the overlying soft tissues. An orthopedic surgeon familiar with the art would understand that the soft tissues would need to be dissected and or retracted out of the region where a cut would be made so they would not be damaged.

FIG. 5 shows three sizes of circular patterns for cuts at the lateral knee joint (501, 502, & 503). (503) designates a cut that would resect substantially the whole lateral joint line. (501, 502) are smaller and of non-specific size. More than one cut 501, 502 and 503 or any combination of (501) and/or (502) cuts can be made at a joint line or bone part interface. The direction of the cut after the entry cut is made can be in any direction substantially radiating from the center of the entry cut in the coronal or x-z plane.

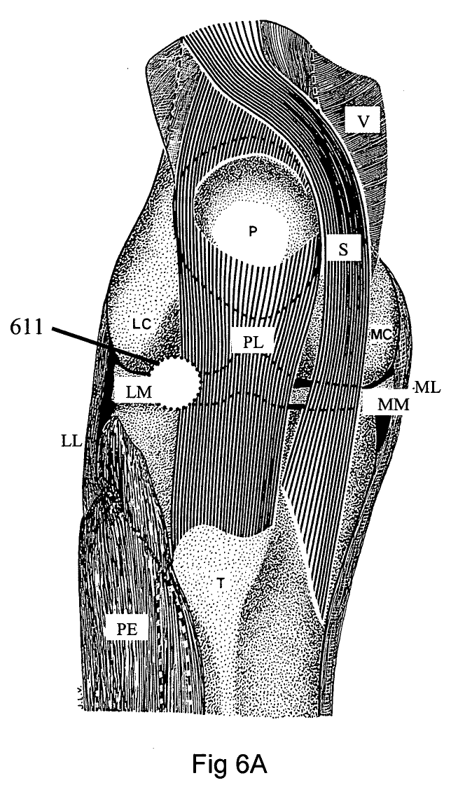

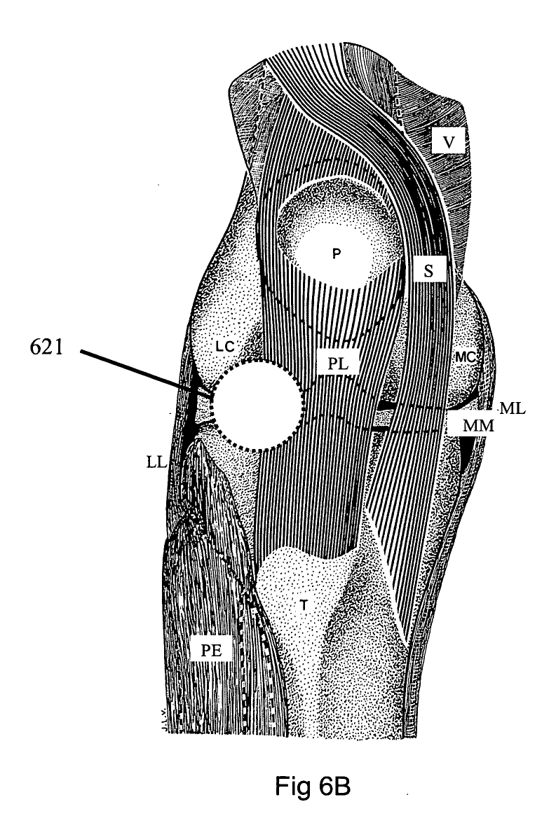

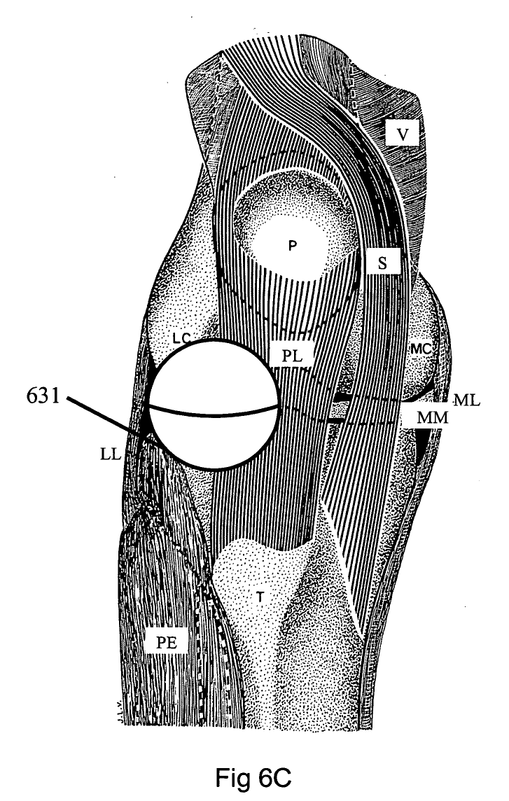

FIG. 6A shows a typical (611) cut at the lateral joint line. The direction of the cut after the entry cut is made can be in any direction substantially radiating from the center of the entry cut in the coronal or x-z plane.

FIG. 6B shows a typical (621) cut at the lateral joint line.

FIG. 6C shows a typical (631) cut at the lateral joint line.

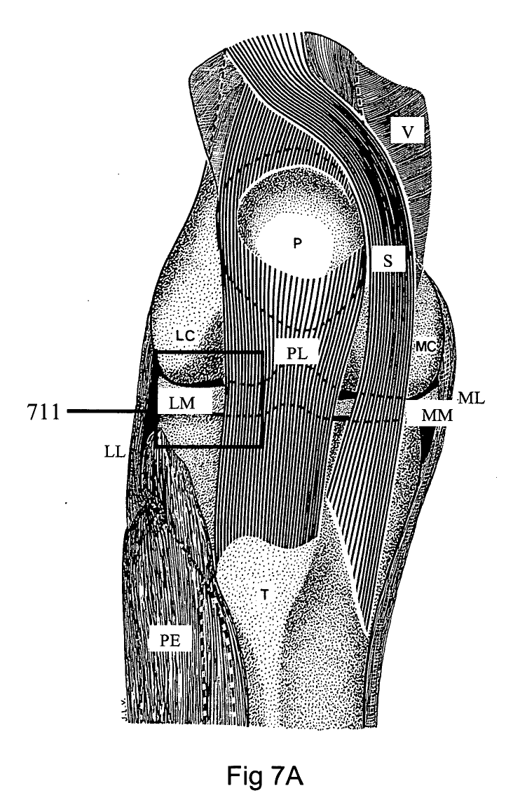

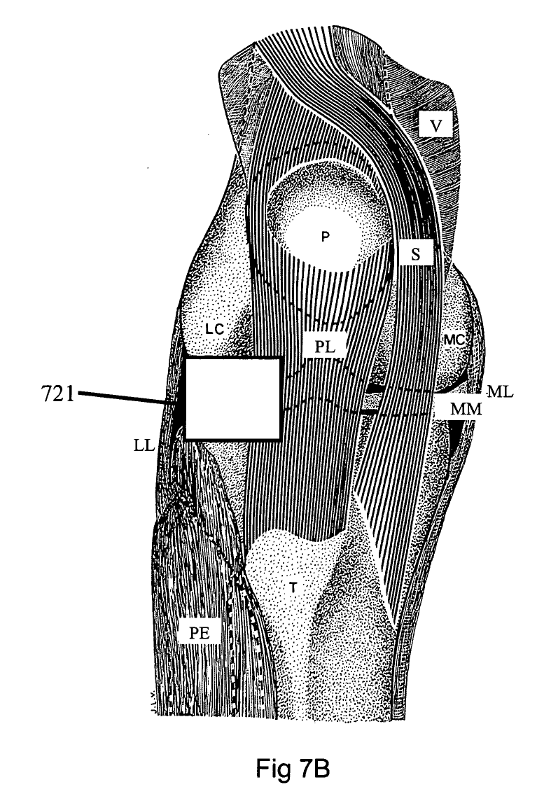

FIG. 7A shows a typical outline for an (711) cut at the lateral joint line. The cut outline is square. It could also be a rectangle or rhomboid. The square, rectangle or rhomboid can have any axis of rotation. The direction of the cut after the entry cut is made can be in any direction substantially radial from the center of the entry cut in the coronal or x-z plane.

FIG. 7B shows a substantially square entry cut (721) without defining the direction of the cut path.

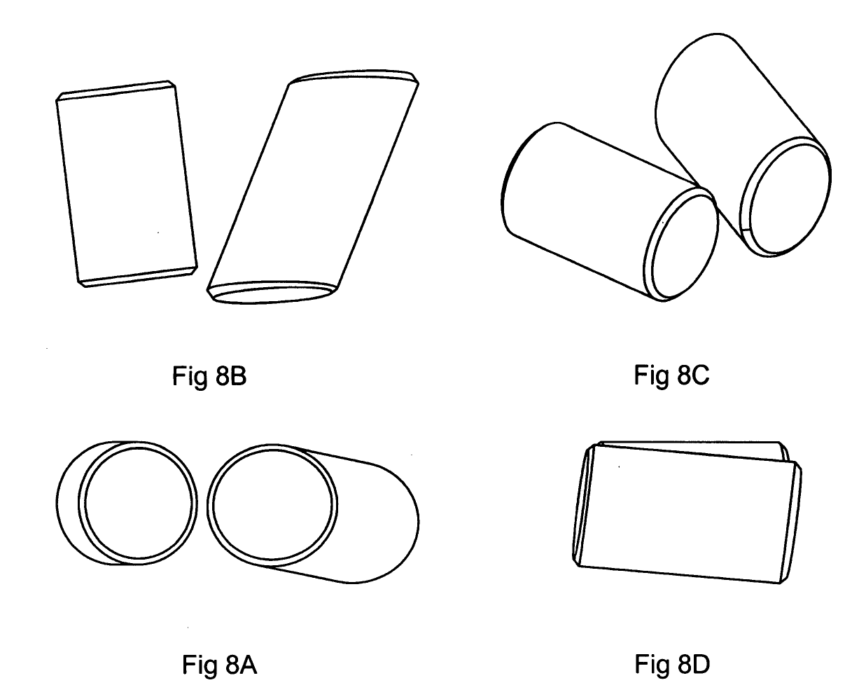

FIG. 8 A-D shows three views of substantially anatomic aligned CrossLink cuts for the knee. The drawings indicate the core or the negative of the cut. FIG. 8A show an AP projection of two CrossLink cuts. The cuts are for a left knee. The medial cut is on the left. The lateral cut is on the right. The medial (left) cut in 8A is directed medial and downward. The lateral cut (right) is directed lateral and downward. 8B shows the degree of medial and lateral diversions of the medial and lateral cuts. 8D show the degree of the downward direction for both the medial and lateral cuts. These cuts in FIG. 8 closely match the normal knee anatomy. Variations in the cut directions can be made for individual patient variations in anatomy or pathology. Each cut can be made to radiate from the center of the entry cut.

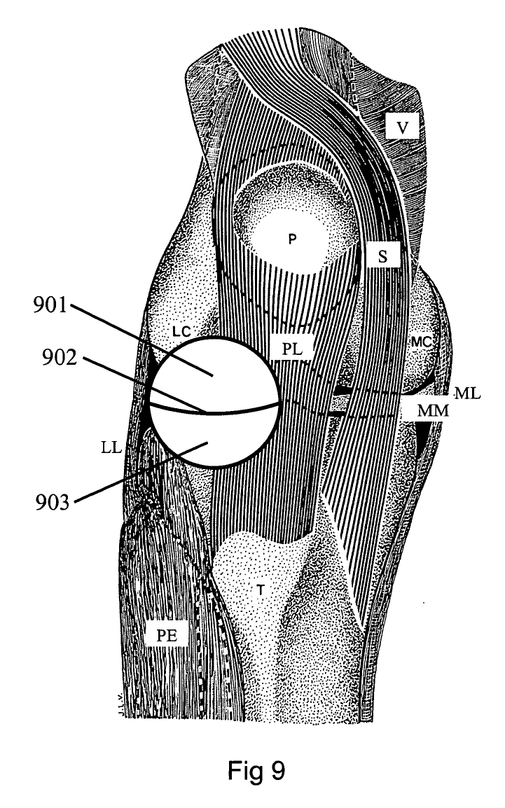

FIG. 9 shows a general implant for a lateral joint line treatment. (901) is the femoral component, (902) is the restored joint line and (903) is the tibial component. This implant has a thicker femoral portion and a proportionately smaller tibial portion.

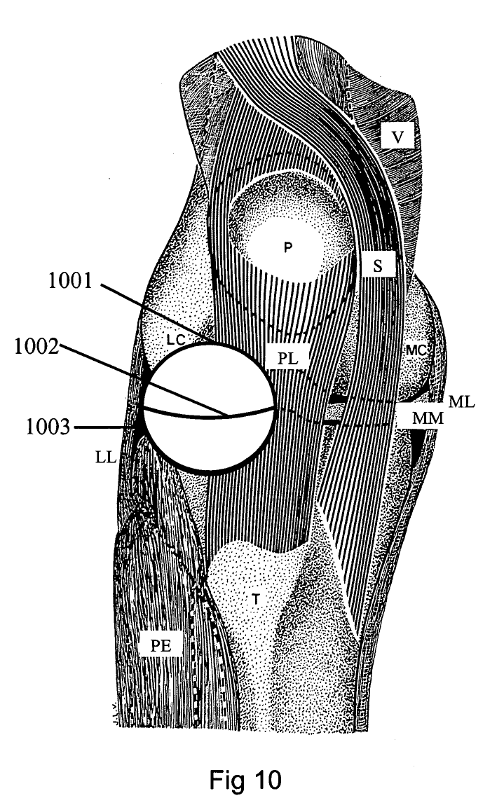

FIG. 10 shows a different general implant for a lateral joint line treatment. (1001) is the femoral component, (1002) is the restored joint line and (1003) is the tibial component. This implant has a femoral portion and a tibial portion that are substantially the same size.

Variations of the position of the joint line in the component similar to FIG. 9 and FIG. 10 can be used to correct joint line height and orientation.

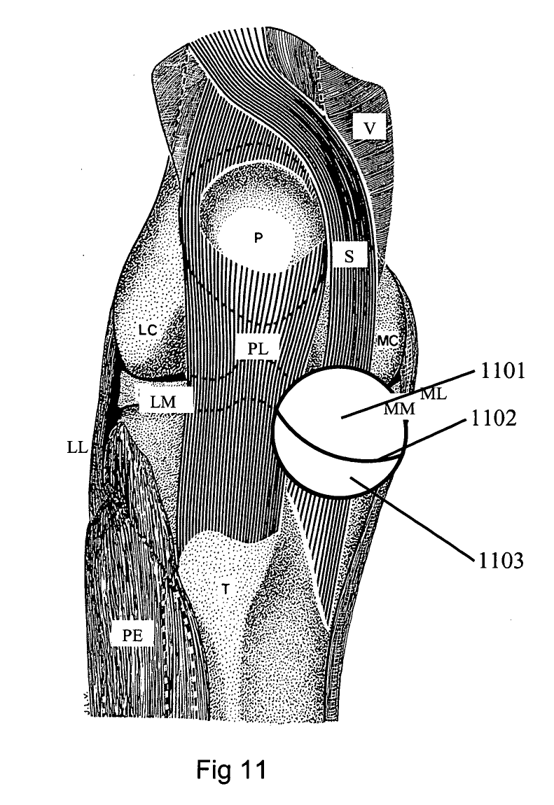

FIG. 11 shows a medial joint line in varus with the leg placed in a valgus stress to restore the lateral joint line to a normal position. (1101) is the top portion of the cut. (1102) is the deformed joint line and (1103) is the deformed medial tibia plateau. FIG. 11is also the position of the cut to treat the varus by osteotomy or implant.

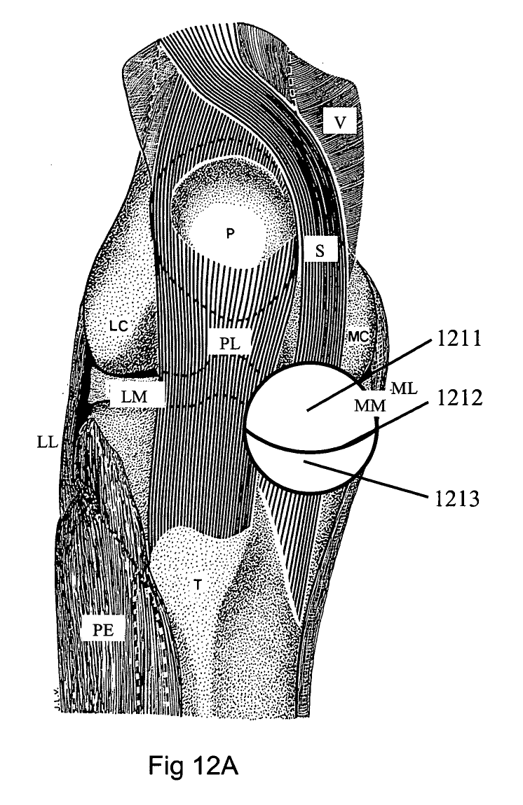

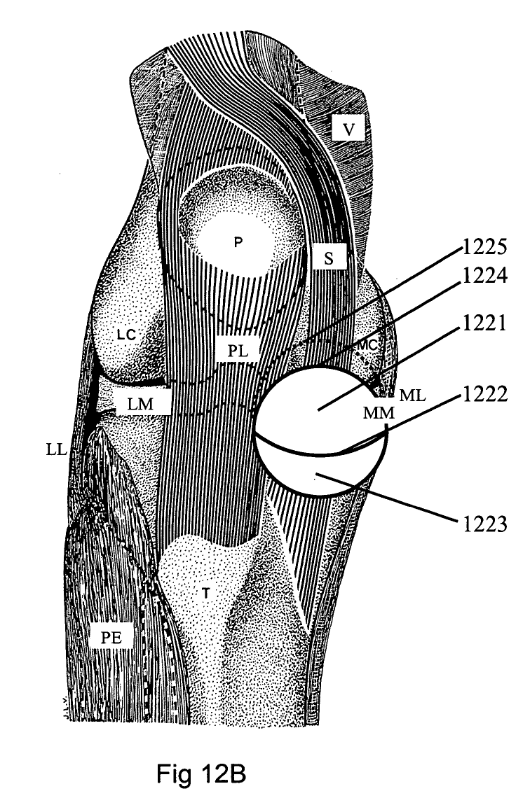

FIG. 12A shows the cut in FIG. 11rotated after the cut to correct the orientation of the joint line (1212).

FIG. 12B is a secondary cut outline for the femur to remove a bone graft and allow elevation of the joint line (1225).

FIG. 12C show the elevation of the joint line and insertion of the bone graft from the femur in the tibia below the initial core, re-establishing the joint line (1234).

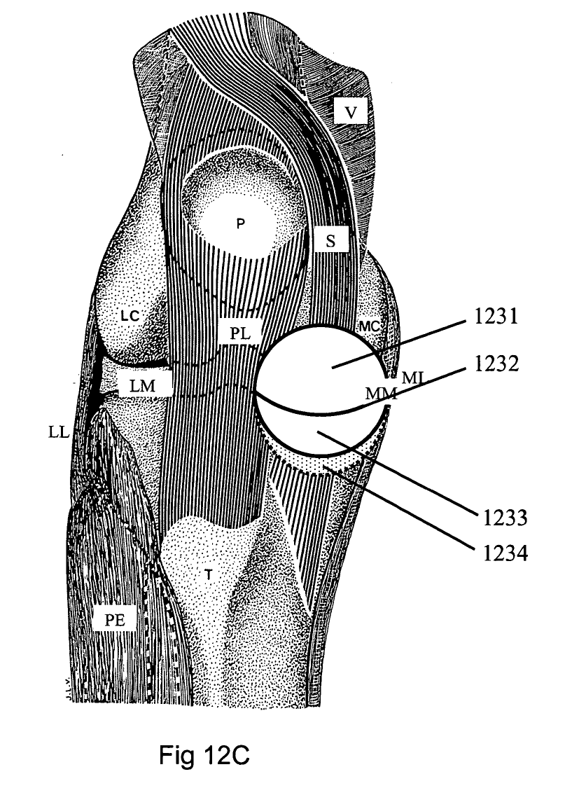

FIG. 13 shows another method in which an implant is used to correct the varus deformity. (1301) is the femoral component, (1302) is the joint line and (1303) is the tibial component.

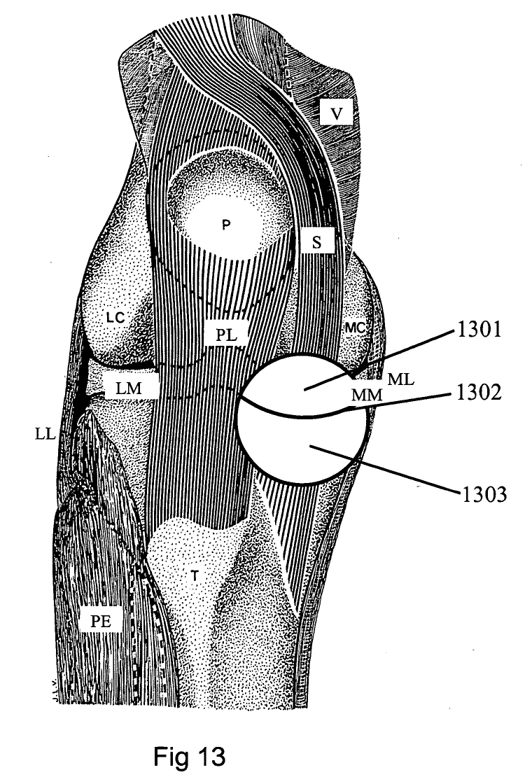

FIG. 14 shows a cutting guide shaft with a tissue protector. The tissue protector protects the soft tissue and neurovascular structures at the back of the knee. The tissue protector can be expanded or inflated.

(1401 is the tissue protector inlet, 1402 is the metal shaft over the inlet to the tissue protector. 1403 is the tissue protector. 1404 is the hard material deployed in a radial pattern fibers to stop the cutting edge from cutting the tissue protector)

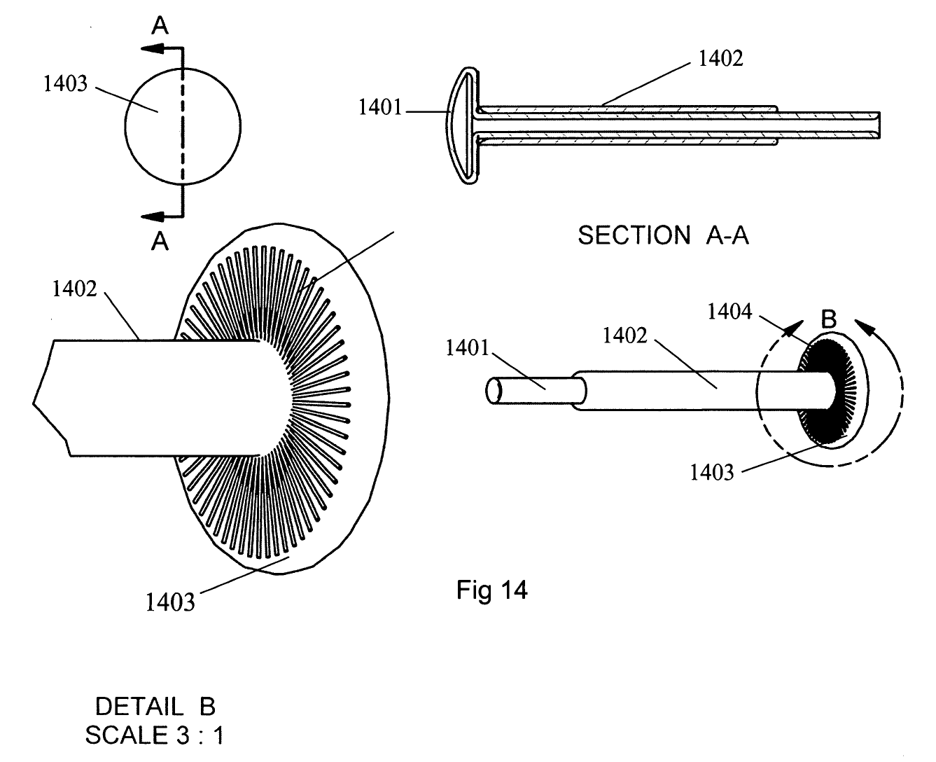

FIG. 15 shows a detail of the expandable portion of the tissue protector (1501) is the tissue protector inlet, (1502) is the hollow portion of part (1501) and (1503) is the expandable tissue protector.

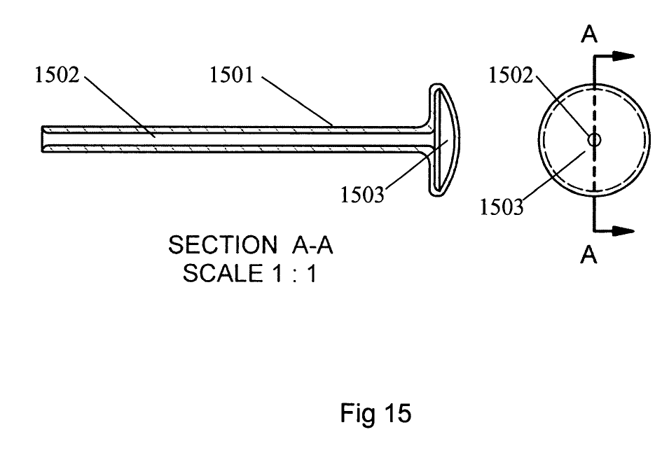

FIG. 16 is another embodiment of a tissue protector/guide pin. (1601is the expandable portion in can be shaped such that it will be efficient in moving or dissecting the soft tissue and neurovascular structures. 1602 is a hard material collar that stops the cutting device before it reaches the tissue protector, 1603 is a hard material sleeve that can act as a guide pin, 1604 is the extension of the tissue protector to deploy the tissue protector, 1605 is the hollow portion of the extension.)

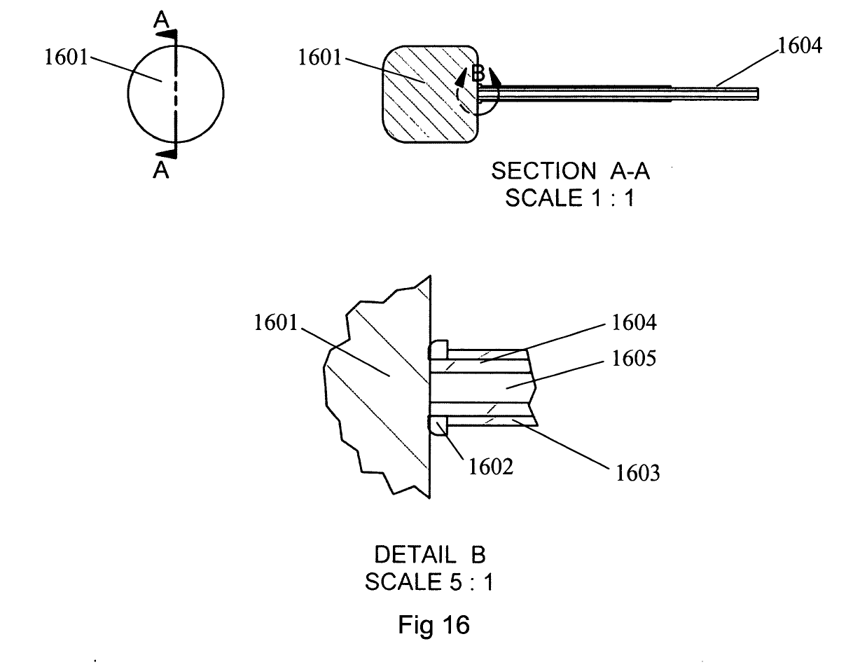

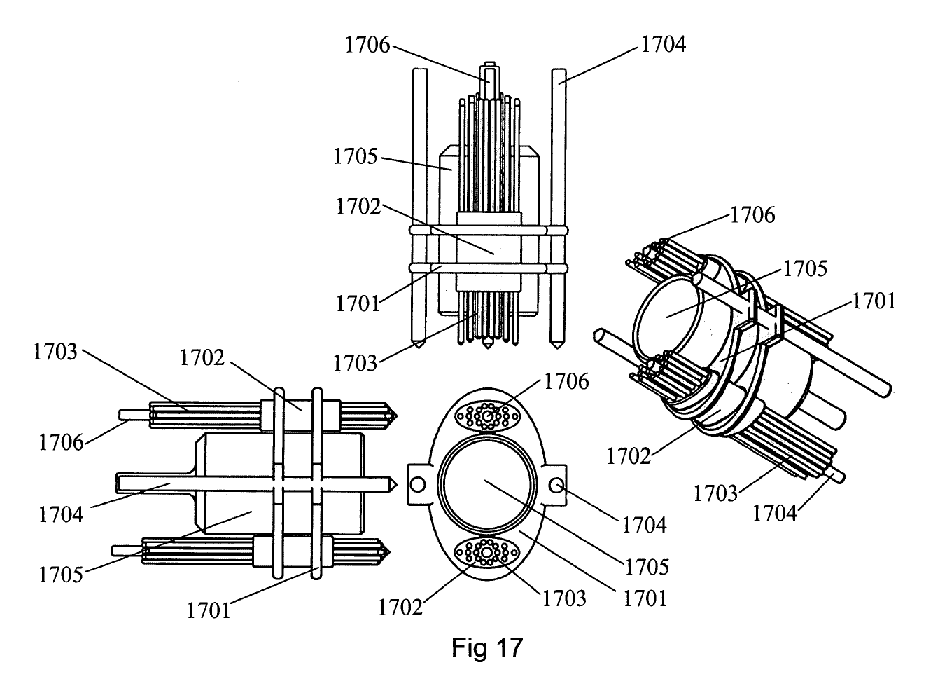

FIG. 17 is an embodiment of a guide mechanism to fix to the bone and control a core cutter or a drill, bit, bore, etc. (1701) is one panel of the housing. The housing is shown with two separated thin flat panels. This enables the cutting device to be positioned in a smaller incision as the first or most forward panel can be on the bone under the tissue and the other panel can remain outside the tissue, allowing the tissue to rest between the inner and outer panels without undue tension on the soft tissue. The panels can be curves especially the front panel. (1702) is a connector piece that connects the two panels. It can be fixed or one or both of the panels can move on the two connector pieces. (1703) are multiple pins that move in the connecting piece. Here 18 pins are shown in each connecting piece.

When the guide mechanism is placed on the femur and tibia the pins adjust or move relative to the housing. They form a negative of the femur and tibia as they engage the bone. Once the pins have conformed to the shape of the bones they are locked into place. After they are locked into place they pins provide several functions. First they stabilize the guide mechanism until the two pins (1704) and the two pins (1706) are placed. The pins (1703) continue to stabilize the guide mechanism after the fixation pins are in place. The pins act as individual probes that are linked with a guidance system such as a computer navigation system. A CN system contains a 3D data base map of the patient's anatomy especially the bone anatomy. Currently a CN system uses a probe with a single point that is placed on the bone and then moved in a fashion such that the infrared sensors correlate the position of the probe tip and the probe with the bone anatomy data in the processor. The device shown provides multiple probes (36) that are also calibrated with respect to each other optically, electrically and/or mechanically to enhance the computer recognition. The combination of each group of 18 pins can only be on a bone in one position with their relative lengths individually displaced to create a negative of the bone anatomy. The distance each pin tip is from the sensor (for example sensors positioned in the connector piece) will be transmitted to a processor and incorporated with information obtained from the probe by more standard current methods. The core cutter shown here is cannulated to go over the previously placed guide pin shaft or it can be fit over another probe or drill bit so that its position can be detected by the CN system.

FIG. 17A is a core cutter (annular cutter). This core cutter is unique in that it has flutes on the outside to decrease friction and remove debris. Flutes can be on the inside of the annular cutter not shown) as well or just on the inside of the annular cutter. The wall can be very thin. Another device allows wall thickness in the range of 0.005-0.001 in or smaller. There is a cannulated centering device or drill bit. (1711 shaft, 1712 cannulated hole in drill bit, 1713 drill bit 1714 thin walled core cutter with flutes on the outside.)

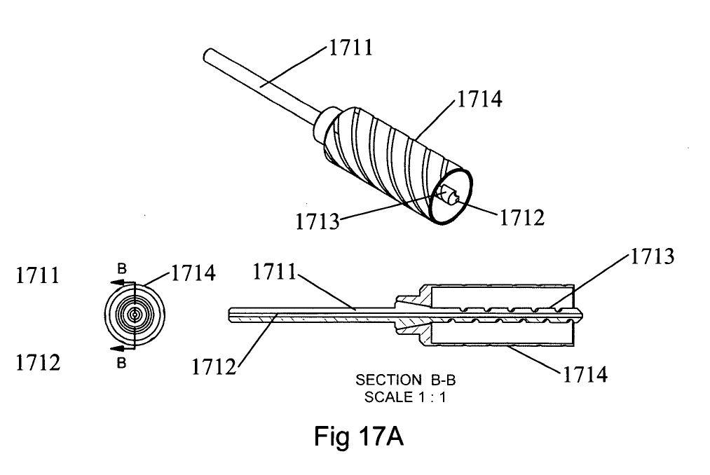

FIG. 18 shows a joint distracter. The distracter can be used on one or both the medial and the lateral sides of the joint to balance joint, evaluate soft tissue constraints or distract the joint. The surfaces of the distracter (1801, 1802) are shaped to match the distal femur and proximal tibia so that the distracter is easy to insert, stable before and during deployment and the distraction force is spread out over a large area. The distracter surfaces (1801, 1802) are made material that is strong enough to tolerate forces but not too rigid or sharp to damage the joint, cartilage or soft tissues. The distracter surfaces can be substantially flexible to allow them to be more conforming to surfaces without damaging them. The distracter can be placed on the well (non-operative) side while the other side is treated or it can be placed on the operative side and cut over.

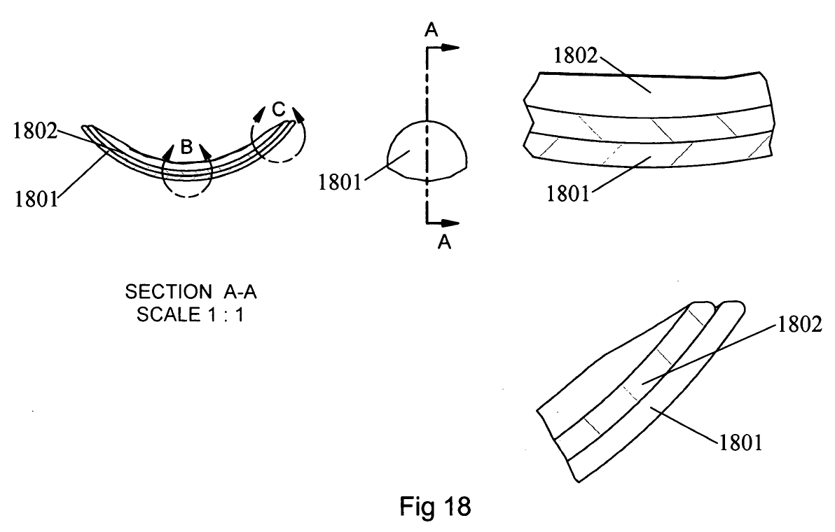

FIG. 19 shows a detail of one embodiment of the distracter. The unit (1903) between the upper (1901) and lower (1902) surfaces separates the surfaces. The mechanism of the unit can have a piston, a telescoping element, a balloon, a mechanical jack or hydraulic jack to force and hold the surfaces apart. The distracter can be calibrated for pressure and timed for duration of application to prevent damage to the soft tissues and cartilage. Typically the distracter is used briefly and intermittently. A miniature version can be place through an incision on one side (operative side) and placed on the opposite side. Similarly the distracter can be placed in the middle of the joint as long as it does not damage the ACL, fat pad or menisci.

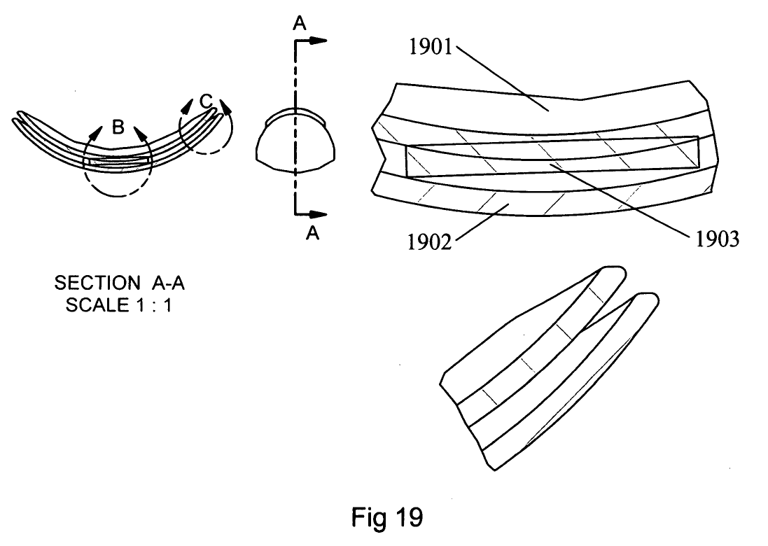

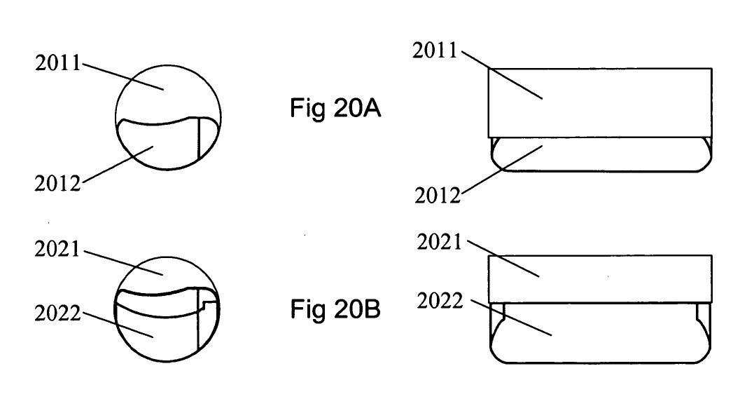

FIGS. 20-33 show some implants specifically for the Unicondylar knee. General implants for other joints and those used in other orthopedic treatments of other bones will be incorporated in this application by association of the methods, instrumentation and fixation elements of this Unicondylar application.

FIG. 20 shows a simplified joint prosthesis that can be use in a knee or any other appropriate joint. For the knee (2011, 2021) would be the femoral component and (2012, 2022) the tibial component. There are curvatures of the surfaces that closely matched the normal knee joint surfaces in both the AP and ML directions similar to current Unicondylar knee replacements.(2011, 2022) differ from (2021, 2022) in that the joint line is at a different level and the amount of femoral or tibia material differs from(2011, 2022) which are nearly the same to (2021, 2022) which are different. By changing the size of the implants and the curvature in the AP and ML directions the basic implant can be adjusted to fit into any joint. Particulars of fixation, shape and size will be built into the design for other joint applications.

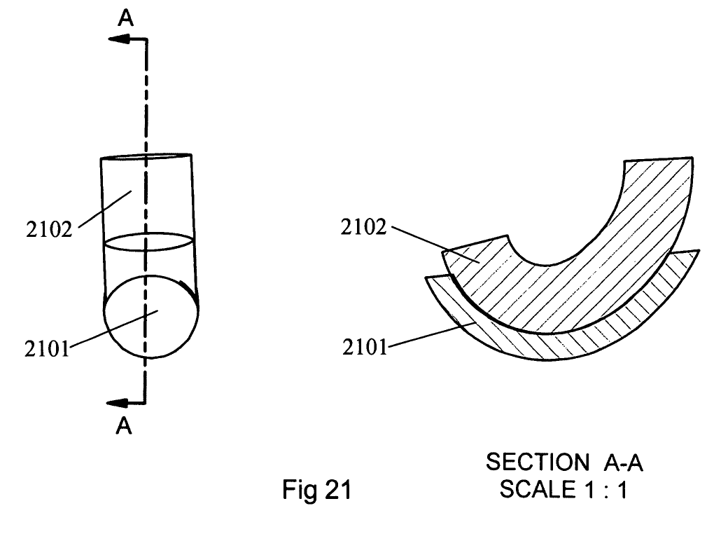

FIG. 21 shows a joint implant (shown for the knee) that is curved in sagittal, coronal and axial planes. The size of the implant and shape of all three curvatures can be designed appropriately for any joint. The guide mechanism or guide pin will need to be designed to aid in the cutting and to account for the curvature. (i.e. curved guide pin and flexible reamers.) Femoral component (2102), tibial component ( 2101).

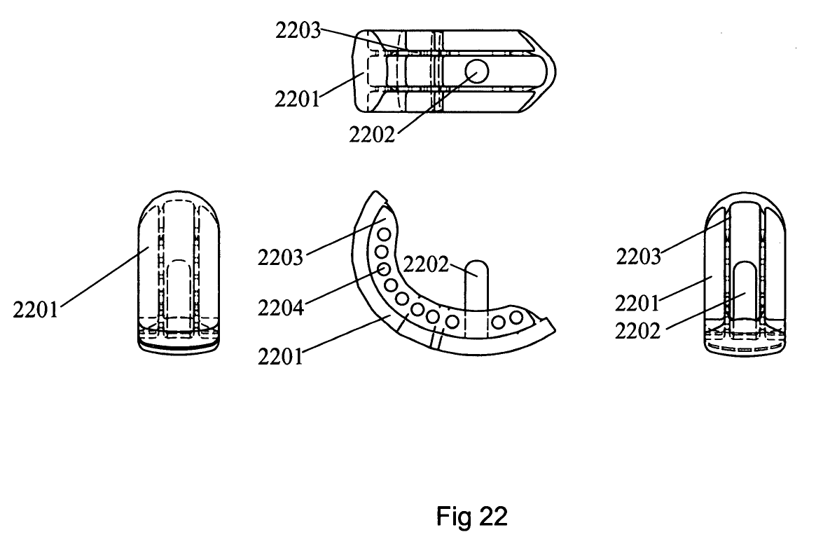

FIG. 22 shows a specific Unicondylar Femoral Prosthesis. It is made to be used with the CrossLink method and instrumentation. The special features include two fins that are deep and have fenestrations for bone in growth. The femoral component can be made of UHMWPE used with a metal tibial tray that has many significant advantages concerning the biomechanics and the tribology of the Unicondylar Knee. These include wear characteristics of the PE, von Meise forces in the PE, plastic deformation, load concentration, thickness of components, tendency of metal tray to re-shape UHMWPE, third body wear, wear particle size, etc. (2201articular surface, 2202 post, 2203 a fin, 2204 fenestration).

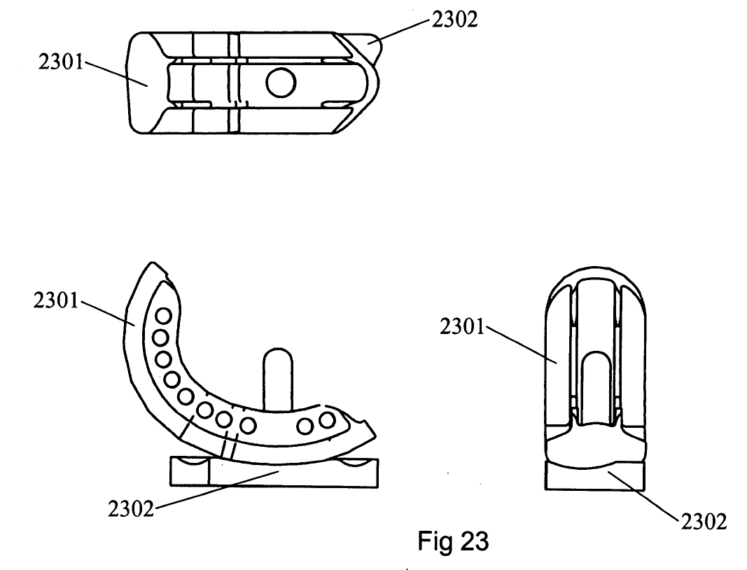

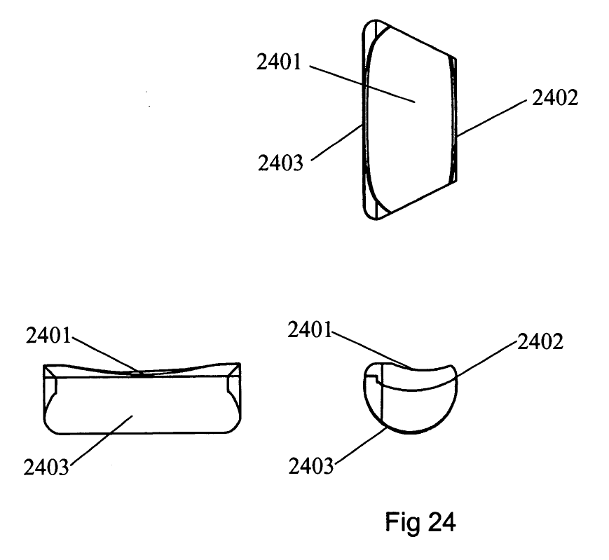

FIG. 23 shows femoral component (2301) from FIG. 22 and a rectangular tibial component (2302). The tibial component is shaped in the axial plane as a truncated triangle (See FIG. 24) with the medial side being shorter. The tibial component is concave to substantially match the AP and ML contours of the femoral component.

FIG. 24 shows a rounded tibial component with a convex upper surface (2401 convex upper surface, 2402 joint line, 2403 body of tibial component).

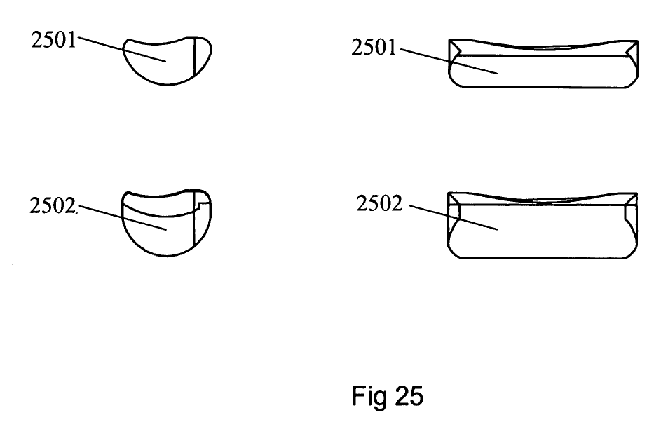

FIG. 25 compares two substantially round tibial components. The diameters are the same. The Joint line is at different levels. (2501 mid joint line, 2502 elevated joint line).

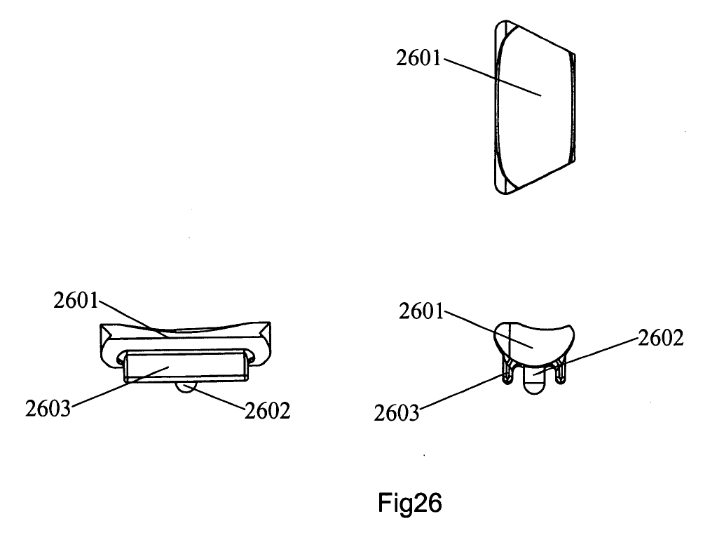

FIG. 26 shows a substantially rounded tibial component with fins and an elongation or post to enhance fixation. (2601 body of tibial component, 2602 post, 2603 fin).

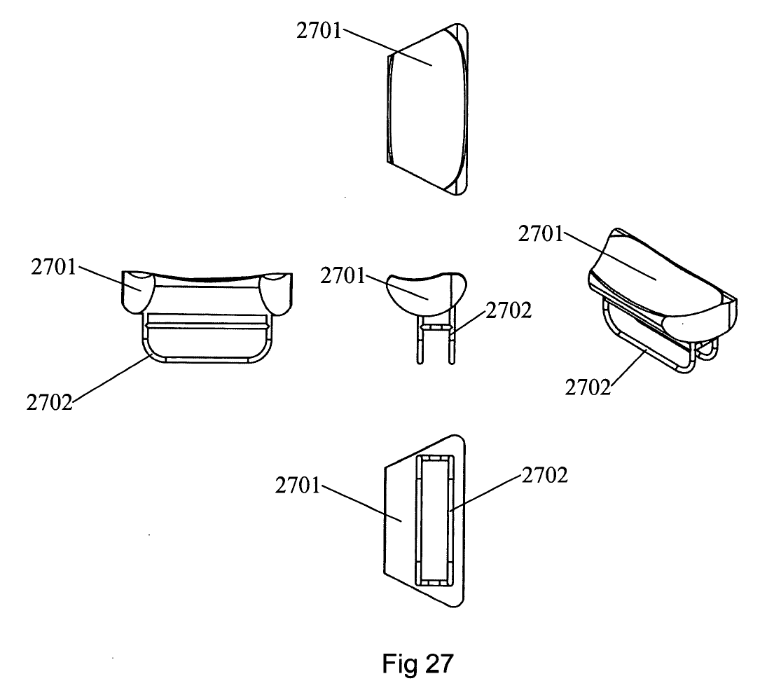

FIG. 27 shows a substantially rounded tibial component (2701) with rebar fixation (2702). The rebar can be in virtually any structural reinforcing pattern that maximizes the fixation to cement and bone. Proper combinations of rebar patterns, materials and ratio of PMMA to rebar volume can approximate bone physical properties more closely than a homogenous material. The rebar can be made of PE, metal or any appropriate material including carbon-based materials. Rebar fixation (2702) can be used in cemented and non-cemented applications. It is very effective in cemented applications especially when there is bone loss requiring cement to fill voids. Rebar fixation can also be used with bone graft or native bone in non-cemented applications.

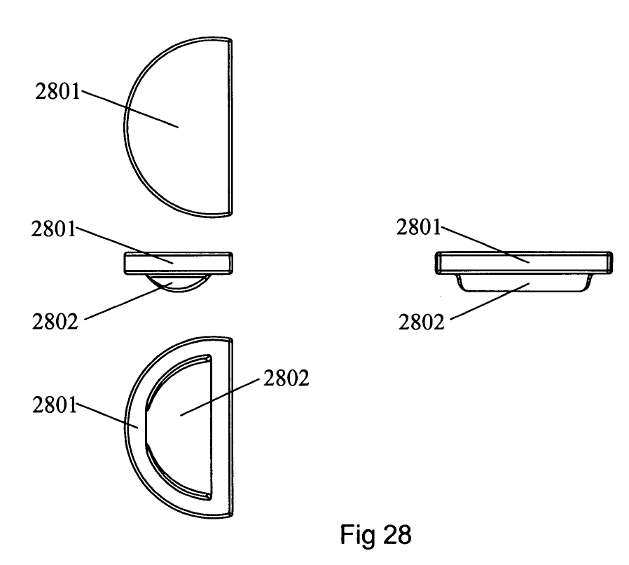

FIG. 28 shows a more conventional shaped tibial component (2801) with a rounded keel (2802). The rounded keel fits in a core or bore made with the CrossLink technique. Additional fixation can be used to add additional stability.

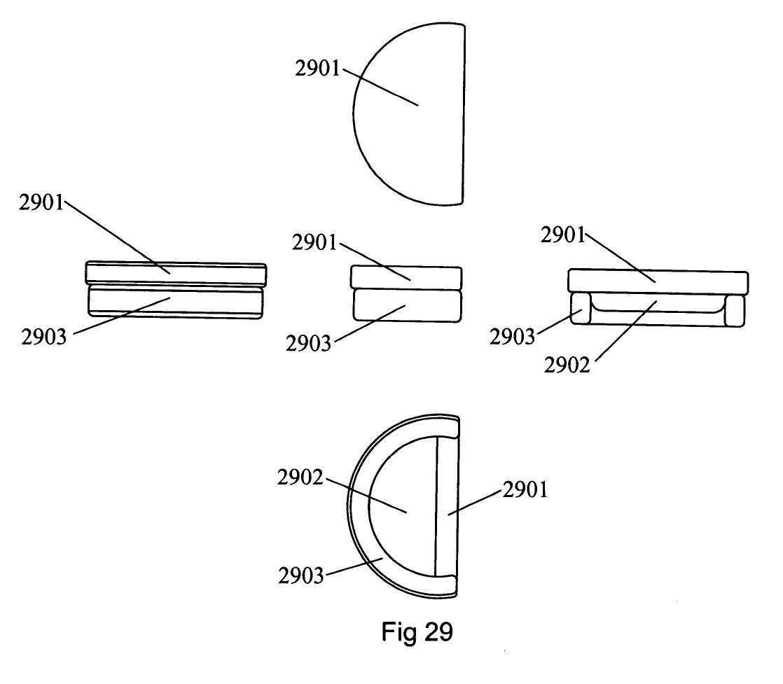

FIG. 29 shows a conventional type of tibial component (2901) with a Magnetic Array to be combined with a mobile bearing with a Magnetic Array (2903) or a femoral component with a Magnetic Array. A mobile bearing with or without a Magnetic Array or Arrays can be placed between a femoral component a tibial component that both have arrays.

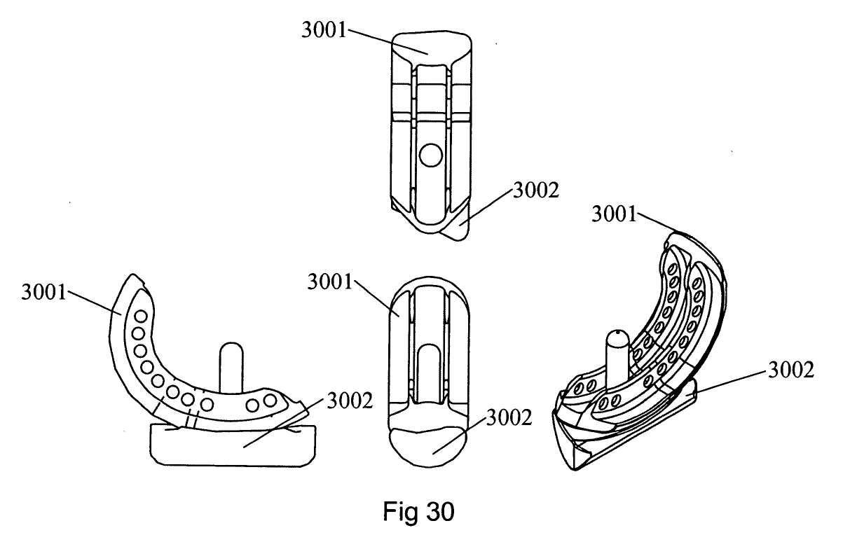

FIG. 30 shows a femoral component (3001) with a substantially round tibial component (3002).

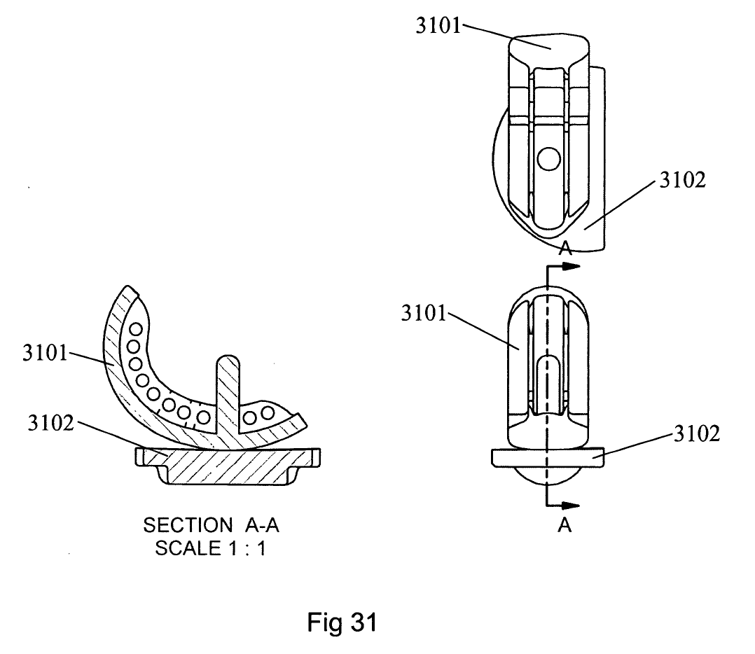

FIG. 31 shows the femoral component (3101) from FIG. 22 and the tibial component (3102) from FIG. 28.

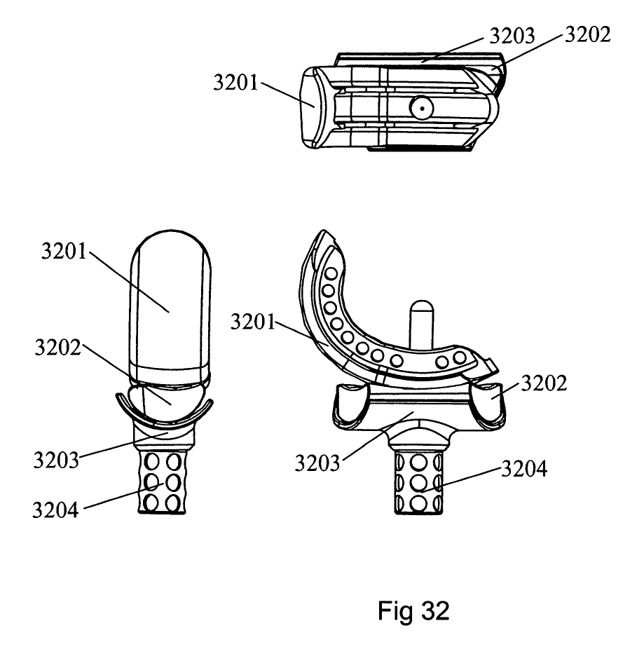

FIG. 32 shows the femoral component (3201) from FIG. 22 with a substantially rounded Mobile bearing (3202) and a substantially rounded tibial component (3204) with fenestrated Crown & Post Fixation (3201femoral component, 3202 mobile bearing, 3203 tibial tray, 3204 fenestrated Crown and Post fixation)

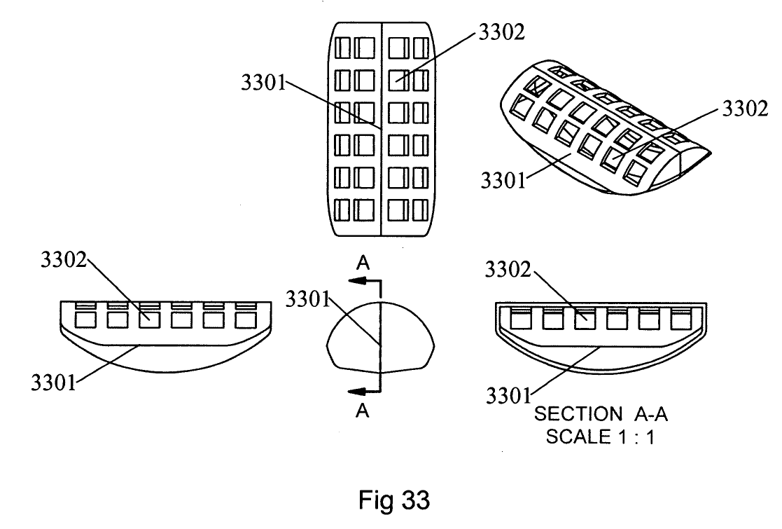

FIG. 33 shows a basic femoral component (3301) with shelled out body and fenestrations (3302) for fixation. Other fixation methods can be added.

1) A method of preparing the adjacent surfaces of a joint for implantation of a joint replacement treatment for joint surface pathology by cutting the proximal and distal sides of the joint using a rotating cutting device
where a first cut is made substantially in one maneuver virtually simultaneously cutting both proximal and distal aspects of the joint such that the part of the cut on one side of the joint is orientated with respect to the part of the cut on the other side of the joint

2) The method of claim 1where the cut is made by an annular cutter

3) The method of claim 1where the cut is made by a drill

4) The method of claim 1where a guide pin is placed in or near the joint to guide the cutting device

5) The method of claim 1where a tissue protector is placed in the joint to prevent soft tissue damage by the cutting device as it is advanced

6) The method of claim 5 where a tissue protector is placed over a guide pin in the joint to prevent soft tissue damage by the cutting device as it is advanced

7) The method of claim 1where a cutting guide is positioned over the joint that rigidly provides a passage for the rotating cutting device in a desired position to cut both sides of the joint substantially simultaneously

8) The method of claim 1where a distraction device is inserted into the joint that is being treated

9) The method of claim 8 where a distraction device consists of two surfaces shaped to substantially match the superior and inferior contours of the joint and has a distraction mechanism to distract the two surfaces

10) The method of claim 8 where the distraction device can assist in correcting mal-alignment of the joint before the first cut is made

11) The method of claim 8 where the distraction device is inserted into a bicondylar joint on the same side or condyle where the surface treatment will be implanted

12) The method of claim 8 where the distraction device is inserted into a bicondylar joint on the opposite side or condyle from where the surface treatment will be implanted

13) The method of claim 8 where the distraction device is inserted into a bicondylar joint on both condyles of the joint where the surface treatment can be employed one or more than one side

14) The method of claim 8 where the distraction device is inserted into a bicondylar joint between the condyles of the joint where the surface treatment can be employed one or more than one side

15) The method of claim 1where the distraction device is external to the joint

16) The method of claim 1where at least two cuts, each cutting both the proximal and distal sides of the joint simultaneously, are made in substantially the same direction

17) The method of claim 1where the joint can be reoriented after the first cut in the joint is made and another cut can be made in substantially a different direction with respect to the first cut

18) The method of claim 1where the first cut through both sides of the joint can be used to guide additional cuts

19) The method of claim 1where a joint requiring the cutting of more than one surface of the joint can use the first cut of the joint to align at least one second cut on at least one second surface

20) The method of claim 16 where the second cut on the second surface is substantially parallel to the first cut on the first surface

21) The method of claim 16 where the second cut on the second surface is not substantially parallel to the first cut on the first surface

22) The method of claim 16 where the second cut on the second surface is orientated to more substantially follow the normal anatomy of the second surface

23) A device that is positioned substantially on or near a joint to control and direct a rotating cutting device that cuts bones on both sides of a joint substantially in one maneuver and substantially simultaneously for a surface treatment of the joint

24) The device of claim 23 that has at least one part of the cutting guide facing the joint or bone presenting multiple movable pins, rod or elongations to the bone or joint surface that can be advanced or be withdrawn in the cutting guide such that the pin tips can touch the bone or joint in multiple places of one or more bone or joint surface and can form a contour of points described by the ends of the pins that is substantially a negative of the adjacent bone or joint surface or surfaces

25) The device of claim 23 where the surface of points forming substantially a negative of the bone or joint surface or surfaces and can stabilize the cutting guide in its preferred position.

26) The device of claim 23 where the preferred position is guided and verified by a computer guidance system

27) The device of claim 23 where the pins forming the negative surface of the bone or joint can be used to provide information that can be utilized by a device to correlate the position of the cutting guide with reference to the bones or joint especially when the device is part of a computerized guidance system

28) The device of claim 24 that has a mechanism that can lock the pins such that the negative of the bone or joint surface made by the pin points is made rigid and maintained

29) The device of claim 24 that has secondary pins, rods or elongations that can be inserted into the bone or joint surface to improve fixation of the cutting guide

30) A deployable device inserted into a joint prior to cutting that protects tissues that are not to be cut especially at the final portion of the cut as it is substantially completed

31) The device of claim 30 that is inserted into a joint over a guide pin or rod

32) The device of claim 30 where the device is inserted to deploy on the opposite or far side of the joint from where the cut was started

33) The device of claim 30 that is deployed by distention

34) The device of claim 33 that incorporates a bladder or balloon as part of the distension mechanism

35) The device of claim 30 deployed hydraulically

36) The device of claim 30 deployed pneumatically

37) The device of claim 30 that is made of a material that resists cutting by a cutting device

38) The device of claim 30 that is reinforced by materials that resist cutting by a cutting device

39) The device of claim 30 that has a mechanical stop preventing a cutting device from contacting the deployed portion
